(19) United States
(12) Patent Application Publication
(10) Pub. No.: US 2026/0227414 A1
(43) Pub. Date: Aug. 6, 2026

(54) ISOLATION OF PODOCYTES FROM SAMPLES

(71) Applicant: SAVRAN TECHNOLOGIES, INC., Newton, MA (US)

(72) Inventors: Yuan Zhong, Wayland, MA (US); Edward Francis Boumil, New Ipswich, NH (US)

(21) Appl. No.: 19/154,737

(22) PCT Filed: Feb. 9, 2024
(86) PCT No.: PCT/US2024/015260

§ 371 Date: Aug. 7, 2025

Related U.S. Application Data
(60) Provisional application No. 63/444,327, filed on Feb. 9, 2023.

(51) Int. Cl. G01N 33/68; G01N 1/30; G01N 1/34; G01N 33/543

(52) U.S. Cl. CPC G01N 33/689; G01N 1/30; G01N 1/34; G01N 33/54326; G01N 2800/368

(57) ABSTRACT

The disclosure relates to methods of isolating podocytes from biological samples such as urine and methods of determining a risk of a pregnant woman developing preeclampsia during the pregnancy. In some embodiments, the disclosure provides methods of isolating podocytes from a biological sample; adding a plurality of antibodies to the biological sample; incubating the biological sample for a time and under conditions sufficient for the antibodies to bind to any podocytes in the biological sample; adding to the biological sample a plurality of magnetic beads; and flowing the biological sample containing the plurality of magnetic beads through a microfluidic chamber within a system that applies a magnetic force in a sufficiently perpendicular direction to the flow to attract the bead-bound podocytes onto a surface of a microchip present in the fluidic chamber.

### CROSS-REFERENCE TO RELATED APPLICATIONS

This application claims priority to U.S. Provisional Application No. 63/444,327, filed on Feb. 9, 2023. The disclosure of the prior application is considered part of and is incorporated by reference in the disclosure of this application.

### TECHNICAL FIELD

This invention relates to isolation of rare cells, and more particularly to isolation of podocytes from biological samples.

### BACKGROUND

Hypertensive complications of pregnancy can be divided into several different categories: preeclampsia, chronic hypertension, preeclampsia superimposed on chronic hypertension, and gestational hypertension. Preeclampsia is a high blood pressure complication that is defined as developing at or after 20 weeks of a pregnancy, that leads to negative health outcomes for both mothers and infants, and if left untreated, can be fatal. Preeclampsia affects 3-7% of pregnancies in the U.S., and can increase pregnancy costs by >50%. The financial burden of preeclampsia in the U.S. alone is over $1 billion for mothers and $1.15 billion for infants (annually) within the first 12 months following birth. The treatment options for preeclampsia are poor where the only true treatment is delivery, though there are several interventions possible with early diagnosis.

Superimposed preeclampsia is a form of preeclampsia that can complicate hypertension of another cause, e.g., chronic hypertension. Women with hypertension associated with diabetes, pre-existing autoimmune disorders, and kidney disease also have an increased chance of developing superimposed preeclampsia.

Given its prevalence, there is a growing need to detect preeclampsia and superimposed preeclampsia earlier to enable early intervention, close monitoring, and proactive treatment. Typical symptoms of preeclampsia are hypertension, elevated urine protein (i.e., proteinuria), edema, and reduced platelets (thrombocytopenia). Currently, blood tests for fms-like tyrosine kinase (sFlt) and placental growth factor (PIGF) are used to detect and predict preeclampsia. However, the predictive values of these tests are low (around 10% or less). Alternative tests that are both non-invasive and offer high predictive power are in urgent need.

Podocytes are a type of specialized epithelial cell that contribute to the structure and function of the kidney glomerulus. In situations of hypertension, podocytes can be forced into the newly filtered urine by the high pressure of the bloodstream, resulting in a state referred to as podocyturia (elevated urinary podocyte levels). Research has found a correlation between the number of urinary podocytes during pregnancy and preeclampsia risk, as well as severity. This research enumerated urinary podocytes by means of culturing isolated podocytes on slides and immunostaining them fluorescently (see, e.g., U.S. Pat. No. 9,810,595), as well as using flow cytometry (Garovic et al., “Flow cytometry as a novel method for detection of podocyturia in preeclampsia,” Am J. Obstet. Gynecol., 206(1):S349 5350, Abstract 794 (2012)). These methods are generally slow and low-throughput and are not useful for detecting low numbers of cells, especially at levels found early in pregnancy.

### SUMMARY

The current disclosure is based, at least in part, on the discovery that even small numbers of podocytes can be isolated from biological samples, such as samples of urine, blood, saliva, semen, mucus, ascites, and/or pleural effusions, of subjects, e.g., pregnant women, e.g., pregnant women patients, suspected of having preeclampsia using novel high-throughput methods and devices to isolate low numbers of podocyte cells with the result that preeclampsia can be detected far earlier than previously possible. The earlier diagnosis can enable caregivers to carefully monitor both the pregnant woman and her fetus and to treat the pregnant woman to reduce symptoms, e.g., with medications and/or bed rest to lower blood pressure and increase blood flow to the fetus, with medications to treat possible seizures, with aspirin prophylaxis, and with steroids to help speed maturation of the fetus's lungs.

In one aspect, the disclosure provides methods of isolating podocytes from a biological sample, e.g., urine, the methods including collecting a biological sample from a pregnant female; adding a plurality of antibodies to the biological sample, wherein the antibodies bind specifically to podocytes that may be present in the biological sample and are bound to a first binding partner of a binding pair; incubating the biological sample for a time and under conditions sufficient for the antibodies to bind to any podocytes in the biological sample; adding to the biological sample a plurality of magnetic beads, wherein the magnetic beads are bound to a second binding partner of the binding pair; and flowing the biological sample containing the plurality of magnetic beads through a microfluidic chamber within a system that applies a magnetic force in a sufficiently perpendicular direction to the flow to attract the bead-bound podocytes onto a surface of a microchip present in the fluidic chamber, thereby isolating the podocytes from the biological sample. In some implementations, the antibodies can be replaced with other molecules such as aptamers, other proteins, or small molecular weight ligands that bind specifically to antigens or markers on the surface of podocytes.

In another aspect, the disclosure provides methods of isolating podocytes from a biological sample such as urine from a pregnant woman, the method including obtaining a mixture of magnetic beads and one or more different types of antibodies, wherein each of the types of antibodies bind specifically to podocytes, and wherein at least some of the magnetic beads are bound to at least one of the types of antibodies in the mixture, with each of the bound magnetic beads being bound to at least one of the antibodies; combining the mixture of magnetic beads and antibodies with the biological sample from a pregnant woman to form a liquid sample, and incubating the liquid sample for a time and under conditions sufficient for the antibodies to bind to any podocytes that may be present in the biological sample to form podocyte-antibody-magnetic bead complexes; and flowing the liquid sample containing podocyte-antibody-magnetic bead complexes, if any, through a microfluidic channel within a system that applies a magnetic force in a direction generally perpendicular to a direction of flow of the liquid sample with a magnetic field strength sufficient to attract the podocyte-antibody-magnetic bead complexes onto a surface of a microchip present in the fluidic chamber, and flowing remaining liquid sample out of the microfluidic channel, thereby isolating the podocytes from the biological sample. In some implementations, the antibodies can be replaced with other molecules such as aptamers, proteins or small molecular weight ligands that bind specifically to surface markers of podocytes.

In some embodiments of these methods, the antibodies are bound to a first binding partner of a binding pair, and the magnetic beads are bound to a second binding partner of the binding pair, wherein the antibodies are bound to the magnetic beads when the first binding partner binds to the second binding partner.

In some embodiments, the antibodies are bound to the magnetic beads or to the first binding partner via a linker molecule, e.g., polyethylene glycol (PEG), and the linker molecule can have a molecular weight of about 5 kDa to 50 kDa, e.g., 5, 10, 15, 20, 25, 30, 35, 40, 45, or 50 kDa.

In certain embodiments, the antibodies bind specifically to any one or more of podocin, podoplanin, or podocalyxin, which are all podocyte-specific markers.

In some implementations, the mixture of magnetic beads and antibodies can include a first set of magnetic beads that are bound to anti-podocin antibodies, a second set of magnetic beads bound to anti-podoplanin antibodies, and a third set of magnetic beans bound to anti-podocalyxin antibodies.

In some embodiments, the microchip includes through holes (pores) or microwells, each with a through hole, wherein the through holes are smaller in diameter than podocytes, but larger than the magnetic beads, to enable excess magnetic beads to be cleared from the surface of the chip.

In various implementations of these method, the liquid sample containing the podocyte-antibody-magnetic bead complexes, if any, is flowed through the microfluidic channel at a flow rate of about 0.1 mL/min to about 100 ml/min, e.g., 1, 2, 3, 4, 5, 10, 20, 30, 40, 50, 60, 70, 80, 90, or 100 ml/min.

In some embodiments, the methods can further include identifying which cells on the microchip are podocytes. For example, in some implementations, identifying cells on the microchip as podocytes includes labeling the cells with reporter groups, wherein the reporter groups are bound to antibodies that bind specifically to podocytes, and then imaging the reporter groups, e.g., fluorescent markers or dyes.

In certain embodiments, identifying which cells on the microchip are podocytes includes any one or more of: positive staining of cells using a reporter group bound to an antibody that binds specifically to a podocyte-specific marker, e.g., synaptopodin; negative staining of cells using a reporter group bound to an antibody that specifically binds to a white blood cell marker, e.g., CD45; and nuclear staining using a stain to label a nucleic acid, e.g., a fluorescent stain, e.g., DAPI (4′,6-diamidino-2-phenylindole). In some embodiments, the methods include the use of all three of positive staining, negative staining, and nuclear staining.

Once the podocytes are identified, the methods can further include counting the cells identified as podocytes. In some embodiments, cells that are positive for synaptopodin and DAPI and negative for CD45 are counted as podocytes.

In some embodiments, a presence of 3 podocytes/10 mL urine indicates a possible risk that the pregnant woman may have or may develop preeclampsia during the pregnancy. In other embodiments, a presence of 5 podocytes/10 mL urine indicates a high likelihood that the pregnant woman has or will develop preeclampsia during the pregnancy.

In some implementations, the methods can further include preparing the biological sample before adding the mixture of magnetic beads and antibodies. For example, the biological sample can be prepared by removing contaminants, by increasing a concentration of any podocytes in the biological sample, or both.

In some embodiments, the methods can further include analyzing the isolated podocytes, e.g., using any or more of qPCR, RT-qPCR, ddPCR, next-generation sequencing (NGS), or Western blotting.

In another aspect, the disclosure features methods of determining a risk of a pregnant woman developing preeclampsia, the methods including obtaining a biological sample, e.g., urine, which may contain podocytes from a pregnant woman; and counting podocytes in a specific volume of the biological sample, wherein a number of podocytes above a threshold level indicates a possible risk that the pregnant woman may have or may develop preeclampsia during the pregnancy.

In some embodiments, the threshold level is 3 podocytes/10 mL urine. In other embodiments the threshold level is set at 5 podocytes/10 mL urine, wherein this threshold level indicates a high likelihood that the pregnant woman already has or will develop preeclampsia during the pregnancy. In any of these methods, the biological sample can be obtained at or before about 20 weeks of gestational age, e.g., 2, 4, 5, 6, 7, 8, 9, 10, 11, 12, 13, 14, 15, 16, 17, 18, 19, or 20 weeks of gestational age. Of course, the methods can also be used later in pregnancy as well.

In some embodiments of these methods, the podocytes can be isolated from the biological sample by the methods of podocyte isolation described herein.

The methods described here have the potential to revolutionize the diagnosis and management of preeclampsia. The currently available tests suffer from poor predictive accuracy, but the methods described herein are non-invasive, quick, low-cost, scalable, easy to automate, and can detect preeclampsia with high sensitivity and specificity, resulting in high positive screening and predictive value. This, in turn, can significantly help not only in management of the condition, but also in prediction of preeclampsia before an official diagnosis, e.g., as a screening tool to help patients understand the potential of future risks and complications and to help doctors know which patients to monitor more closely as the disease progresses. In addition, the new methods can differentiate between superimposed preeclampsia or preeclampsia and chronic hypertension.

Other embodiments can serve various diagnostic, prognostic, and therapeutic purposes in obstetrics and related fields. In one embodiment, they can be included into the current diagnostic workflow, enhancing the accuracy and reliability of existing screening methods.

The podocyte isolation methods can also serve as prognostic indicators, monitoring the response to treatment in individuals diagnosed with preeclampsia, and providing valuable insights into the progression and severity of preeclampsia in pregnant individuals. By quantifying podocyte numbers and assessing their characteristics, healthcare providers can better anticipate the clinical course of the condition, evaluate the effectiveness of therapeutic interventions, and adjust treatment plans as necessary to optimize patient outcomes. The new methods described herein can also be used to facilitate the development and evaluation of novel therapeutic interventions aimed at mitigating the adverse effects of these conditions on maternal and fetal health.

These new methods of isolating podocytes also hold potential for research applications, enabling the investigation of underlying disease mechanisms and the identification of novel biomarkers and therapeutic targets.

Furthermore, these methods of isolating podocytes hold promise for detection and monitoring of other renal and cardiovascular conditions beyond preeclampsia, including, but not limited to, chronic kidney disease (CKD), diabetic nephropathy, glomerulonephritis, and cardiovascular diseases like heart failure and atherosclerosis.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic of a system that can be used to isolate podocytes from biological samples.

FIG. 2 is a schematic of a process as described herein, in which a podocyte is captured on an antibody-bead complex, and showing the steps of the process.

FIG. 3A is a graph showing the number of podocytes isolated from actual urine samples from pregnant women diagnosed with preeclampsia or gestational hypertension versus the number of podocytes in samples from a control group not diagnosed with preeclampsia.

FIG. 3B is a graph showing the number of podocytes in actual urine samples from pregnant women with superimposed preeclampsia (n=6) compared to the number of podocytes in those with chronic hypertension (n=4).

FIGS. 4A and 4B are graphs of the Positive Predictive Value (PPV) and the Negative Predictive Value (NPV) curves, respectively, of the tests described herein, generated against prevalence.

FIG. 5 is graph showing the Receiver-Operating Characteristic (ROC) curve of podocytes for preeclampsia contrasted with the sFlt/PlGF results. The ROC curve analysis was performed and the area under the curve (AUC) was computed to evaluate the predictive performance of the podocyte count as a biomarker for preeclampsia.

FIG. 6 is a graph of isolated podocyte count as a marker of high vs. low risk for preeclampsia. Given the early prediction scenario, a screening cutoff number of 3 podocytes/10 mL urine was determined to clearly separate samples from patients with high vs. low risk.

Like reference symbols in the various drawings indicate like elements.

### DETAILED DESCRIPTION

The new methods of isolating podocytes from samples, e.g., urine sample, are non-invasive and can detect even low numbers of podocytes from samples. Once the podocytes are isolated, they can be counted, and the number of podocytes can be used as a biomarker for preeclampsia. The isolated podocytes can also be analyzed, e.g., genomically, to provide even further information useful in the diagnosis and prognosis of these complications.

Podocyte Isolation from a Biological Sample

In the new methods described herein, a biological sample, such as a urine sample, e.g., a 25-100 mL urine sample, is collected from a pregnant patient. In various embodiments, isolation of the podocytes can begin with different volumes of urine, including, but not limited to, 25, 50, 75, or 100 mL of urine.

In some implementations, the urine sample can be optionally subjected to sample preparation steps that can include various combinations of filtration, purification, and centrifugation to increase the concentration of the typically low number of podocytes that are originally present in a relatively large urine sample (e.g., 75 mL) to a much smaller volume (e.g., 500 μL). The sample preparation steps can also serve to clear out large debris, parts of tissue, or other cells, from the original urine sample that might otherwise interfere with the podocyte isolation process.

More specifically, the sample preparation steps can be accomplished in the following fashion: The urine specimen can be centrifuged and resuspended into buffers like PBS or cell culture media. Supplementations such as fetal bovine serum (FBS) and bovine serum albumin (BSA) with different concentrations can be added into the buffer. In one embodiment, the urine sample can go through filtration before an incubation step using a cell strainer, e.g., a 70 μm cell strainer, to clear the sample from crystals and other large particles. The filtration can be implemented using cell strainers or mesh filters with different mesh sizes from 20 to 100 μm or above. In other embodiments, the urine sample can be filtered either before or after centrifugation and resuspension.

In another implementation, the cells can be fixed after centrifugation and resuspended using chemicals including, but not limited to, 4% paraformaldehyde (PFA) solution, methanol, or acetone.

In general, the sample, e.g., once prepared, is incubated with magnetic (or paramagnetic or superparamagnetic; simply referred to herein as “magnetic particles” or “magnetic beads”) particles functionalized with antibodies, aptamers, other proteins, or small molecular weight ligands that bind specifically to one or more antigens present on the surface of podocytes. Examples of these antigens include podocin, nephrin, neph1-3, podoplanin, podoendin, podocalyxin, P-cadherin, GLEPP-1, TRPC6, and synaptopodin. For example, the magnetic beads and antibodies can form an antibody-bead “cocktail” in which each bead is bound to one type of antibody, and the cocktail can include one or more of a first set of magnetic beads that are each bound to an anti-podocin antibody, a second set of magnetic beads that are each bound to an anti-podoplanin antibody, and a third set of magnetic beans that are each bound to anti-podocalyxin antibodies. Any such cocktail will also contain many magnetic beads that are not bound to any antibodies, but they are removed from the liquid samples by the systems described herein.

In some implementations, the surface of the magnetic particles can be derivatized or coated with one half of a binding pair, such as streptavidin, and the antibody can be linked with the other half of the binding pair, e.g., biotin, to facilitate the antibody-bead binding. In other implementations, the bead-antibody binding can be enabled using other binding methods and binding molecules such as amine-based (NHS) conjugation of proteins to the magnetic beads, or Protein A or Protein G to bind the antibodies to the magnetic beads.

The magnetic bead “cocktail” is then incubated with the sample for a time sufficient to enable the magnetic beads to bind to podocytes that may be in the sample, e.g., the prepared sample, and form podocyte-antibody-magnetic bead complexes. The antibody concentration can be from 0.01 μg to 10 μg, e.g., from 0.25 μg to 2.5 μg

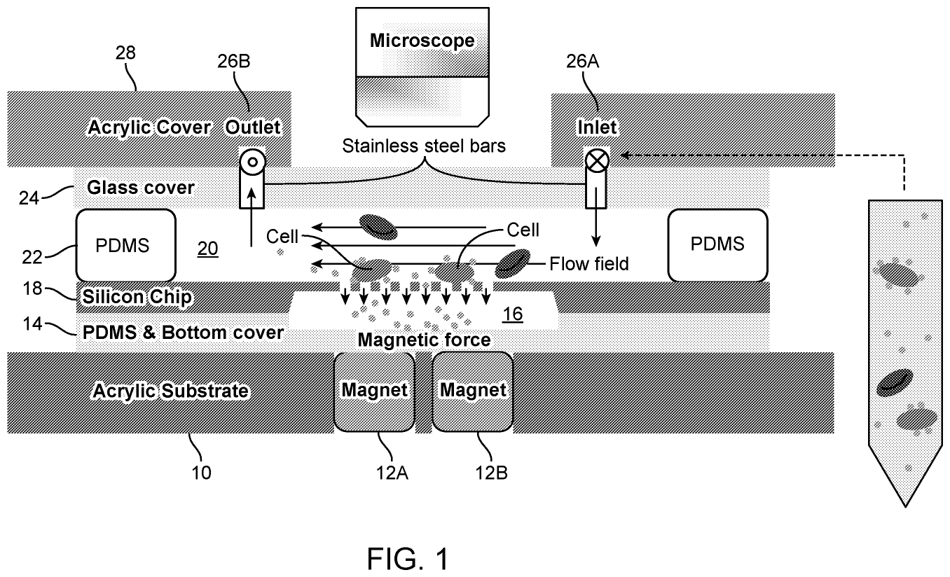

As shown in FIG. 1, in one implementation, the sample (e.g., urine)-bead mixture is then flowed through a microfluidic chamber of a microfluidic system that applies a magnetic force in a sufficiently perpendicular direction to the flow of liquid through the microfluidic chamber to attract the bead-bound podocytes onto the surface of a microchip that is also present in the fluidic chamber. In one embodiment, the microchip, e.g., silicon microchip, has pores, e.g., through holes, that are smaller than podocytes, but larger than the magnetic beads, so as to clear out the excess magnetic beads from the surface of the chip. For a detailed description of microchips that can be used in the new methods, see, e.g., U.S. Pat. Nos. 11,478,797 and 11,077,439, which are incorporated herein by reference in their entireties. In various implementations, the flow rate can be from about 0.1 mL/min to about 100 ml/min, e.g., 0.25, 0.5, 0.75, 1, 2, 3, 4, 5, 10, 20, 30, 40, 50, 60, 70, 80, 90, or 100 ml/min.

As shown in the example of FIG. 1, the microfluidic system includes a substrate 10, e.g., an acrylic substrate, in which, in some examples, one or more, e.g., two magnets 12A and 12B can be embedded. The microfluidic chip can be included in a cartridge, which can include the substrate and magnets, or the cartridge can be inserted into a cavity of a system that includes the substrate and magnets. The cartridge includes a bottom cover 14, e.g., of polydimethylsiloxane (PDMS) and plastic sheet, with a lower chamber 16 that is covered by a microchip 18, e.g., a silicon microchip, which includes micropores (through holes) that are sized to be smaller than podocytes and larger than the magnetic beads. Then a microchannel 20 is created in the cartridge with side walls 22, e.g., a PDMS spacer, and the microchannel is covered with a cover 24, such as a glass cover. The cartridge includes an inlet 26A and an outlet 26B, which can be made, for example, by inserting hollow stainless-steel bars into the microchannel through the side walls or the cover, or other openings can be made. The cartridge can include a cover 28, e.g., an acrylic cover, or that cover can be part of the system that houses the cartridge. The acrylic cover includes an opening to allow visualization of the cartridge using a microscope.

In FIG. 1, the podocytes bound with magnetic beads in a liquid sample are located in a vessel, e.g., test tube, and the liquid sample is passed from the test tube through the inlet and into the microfluidic channel. It then flows out through the outlet, which is connected to a peristaltic pump to generate the flow. As the liquid sample flows (from right to left in the example shown in FIG. 1, the magnetic beads are exposed to a downward magnetic force, e.g., perpendicular to the direction of flow.

This magnetic force and the rate of flow are carefully controlled, e.g., by the operator, or by an automated programmed controller, such that the flow is sufficient to move the liquid sample including any podocytes and magnetic beads through the microchannel, and to enable the unbound magnetic beads to pass through the through holes into the lower chamber. This allows debris and any cells that are not bound to magnetic beads to pass through the microchannel and out the outlet. At the same time, the magnetic force (downward as shown in the example of FIG. 1) is controlled to be sufficient to hold in place (against the force of the liquid sample flowing perpendicular to the magnetic force) any podocytes bound to magnetic beads on the surface of the chip, and to cause unbound magnetic beads to pass through holes into the lower chamber.

The magnetic field can be controlled manually, for example, by the placement of different numbers of specific permanent magnets, or in an automated manner by a system that creates an electromagnetic field of the appropriate field strength at the appropriate time periods. For example, the system can generate the magnetic field by moving permanent magnets closer or further away from the microchannel. For example, two or more neodymium magnets can be used. In other implementations, 1 to 10 or even 20 block magnets, or magnets in other shapes and sizes, can be employed.

The balance of flow rate of the liquid sample and the magnetic field strength also can be maintained by controlling the flow rate of the fluidic pump, which is easier to control and adjust than the magnetic field strength. The appropriate flow rate, e.g., 0.25, 0.5, 0.75, 1, 2, 3, 4, 5, 10, 20, 30, 40, 50, 60, 70, 80, 90, or 100 ml/min, e.g., 0.5 mL/min to 5.0 ml/min, can be determined to achieve the best capturing efficiency and purity. For example, more magnets generally allow higher flow rate, in various implementations, the flow rate can be from about 0.1 mL/min to about 100 mL/min, or more narrowly from 0.3 mL/min to 2.0 mL/min.

Once the entire liquid sample has passed through the microchannel, the unbound magnetic beads have been removed from the liquid sample and held in the lower chamber, any debris and the like have been removed through the outlet, and only the podocytes remain on the microchip, and have thus been isolated. These podocytes can now be counted, e.g., by being immunofluorescent labeled and counted, using the microscope, e.g., an epifluorescence microscope.

In addition, one can flow a clean buffer or other liquid through the microchannel without the presence of the magnetic force to allow the isolated podocytes to be taken up by the buffer and flowed out through the outlet for collection and further analysis, if required.

In another implementation, the specific binding of an antibody-magnetic bead conjugation to podocytes can be accomplished in a two-step method. In step 1, a primary antibody bound to a first binding partner of a binding pair, such as biotin, is incubated under standard conditions to bind specifically to any podocytes in a sample. In step 2, magnetic beads are introduced to bind to the primary antibodies, wherein the magnetic beads are bound to a second binding partner of the binding pair, such as streptavidin, and the two binding partners bind the beads to the primary antibodies.

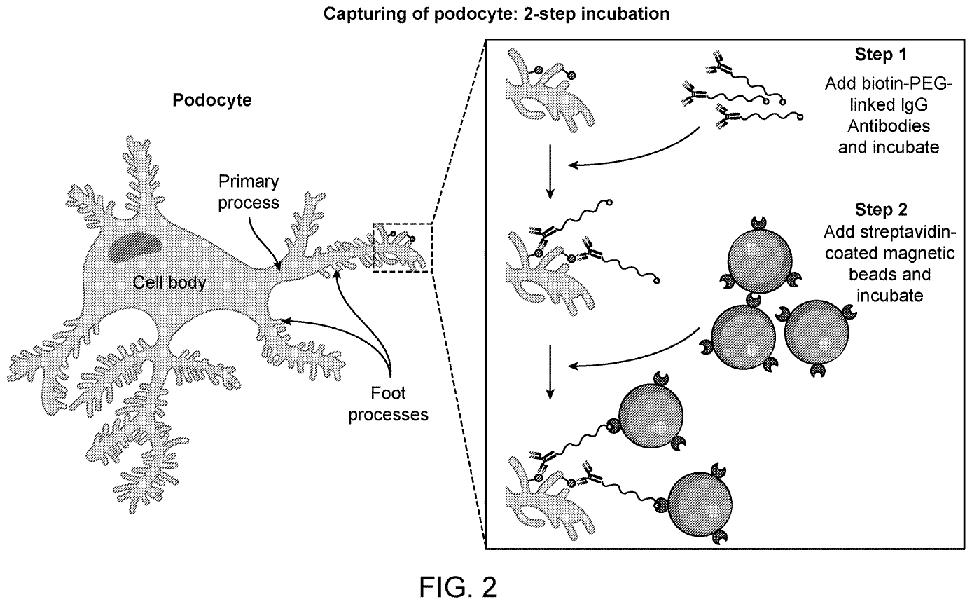

In another implementation as shown in FIG. 2, the primary antibody is bound to a linker, such as a biotinylated PEG linker with a molecular weight of about 5 kDa to 50 kDa, e.g., 5, 10, 15, 20, 25, 30, 35, 40, 45, or 50 kDa (or about 30 nm to about 300 nm), which can be especially useful to link a magnetic bead to an antigen that is present in a relatively “deep” or far location within the cell surface, to which it might otherwise be difficult to bind to the magnetic bead because of the presence of numerous primary and “foot” processes that extend from the podocyte cell bodies.

Counting and/or Analysis of Captured Podocytes

Once captured, the podocytes can be counted and/or analyzed. For example, the podocytes captured on the microchip surface can be counted by fluorescent immunostaining with an epifluorescence microscope or automated, high-throughput fluorescence imaging devices. In one iteration, podocytes can be identified by antibodies that are bound to a reporter group, such as a fluorescent marker, e.g., 6-Ccrboxyfluorescein (6-FAM), VIC®, ABY®, NED®, SUN®, JUN®, Cy3, Cy5, and Cy5.5. These antibodies can include podocyte-specific cell surface markers including podocin, nephrin, neph1-3, podoplanin, podoendin, podocalyxin, P-cadherin, GLEPP-1, TRPC6 and synaptopodin. Podocytes can be imaged either live or after being fixed with paraformaldehyde. For applications where podocytes are fixed and/or permeabilized, antibodies can also include intercellular markers such as vimentin, WT-1 and CD2AP.

In some implementations, different antibodies than the ones used in the antibody cocktail conjugated to the magnetic beads for capturing, can be used to label and image the captured podocyte cells. In other implementations, negative selection, e.g., using fluorescent labeling of white blood cell can be employed to exclude these WBCs from podocyte identification. Specific antibodies against markers such as CD45, CD2, CD11b, CD14, CD16 and CD138 can be utilized for this purpose.

In another implementation, nuclear specific dyes, such as DAPI (4′,6-diamidino-2-phenylindole), Hoechst, propidium iodide (PI), and SYTOX, can used to label and visualize nucleated cells for podocyte identification.

Alternatively, podocytes can be identified using fluorescence in-situ hybridization via targeting DNA sequences or podocyte-specific mRNA expression. Fluorescent nucleic acid probes can be directed against podocin, nephrin, neph1-3, podoplanin, podoendin, podocalyxin, P-cadherin, GLEPP-1, TRPC6 synaptopodin, and vimentin.

For example, in some embodiments, the captured podocytes are positively identified by immunofluorescent staining, using one or more of the following steps:
(1) positive staining of podocyte-specific markers, e.g., using antibody dye targeting synaptopodin, a specific podocyte cell marker;
(2) negative staining of white blood cells, e.g., using an antibody dye targeting CD45, a white blood cell marker; and
(3) nuclear staining of nucleic acids such as DNA, e.g., using DAPI (4′,6-diamidino-2-phenylindole) or other fluorescent stain for DNA or RNA.

One then counts the cells that are positive for one or more of, e.g., all three of, synaptopodin and DAPI, and negative for CD45, as podocytes. These steps are important, as there can be hundreds to thousands of other non-podocyte cells non-specifically captured on the microchip, and thus a combination of positive, negative, and nuclear staining provides the highest level of identification accuracy. For example, these background cells are mostly white blood cells and red blood cells or platelets, and the white blood cells are ruled out by anti-CD45, and the red blood cells or platelets are ruled out by nucleated staining, because they lack nuclei.

In another implementation, the captured podocytes can be analyzed and/or enumerated by means of one-step RT-qPCR, composed of a warm-start reverse transcriptase cDNA synthesis step followed by hot-start Taq DNA polymerase qPCR. Here, the podocytes can be washed out of the chamber and segmented into one or more PCR plates which include (but are not limited to) 96-, 384-, and 1536-well plates or custom-made well plates with greater number of wells to single cell levels (each well contains only one podocyte, or no podocytes). In such microwell plates, given the low numbers of podocytes, there may be many empty wells.

Podocytes can then be enumerated using either genetic (genomic DNA) markers or by cell-specific mRNA markers by counting the number of positive wells for podocyte-specific markers. These podocyte-specific mRNA markers can include, but are not limited to, primer/probe sets capable of detecting expression of nephrin, podocin, podocalyxin, synaptopodin, and podoplanin mRNA. For example, primers, e.g., commercially available primers, can be designed to be exon-spanning to prevent genomic DNA amplification. Primers can be 15 to 30, e.g., 19 to 25, e.g., 20, nucleotides in length. In one implementation, primer concentrations can be 2.5 μM, however, in other implementations primer concentrations can be 0.5, 1, 1.5, or 2 μM. DNA primers can be composed of standard deoxyribonucleic bases or a mixture of locked-nucleic acid modified bases and standard deoxyribonucleic bases. Probe concentrations can be 2.5 μM, however in other embodiments they may be 1, 1.5, 2, or 3 μM.

Probes may feature QSY, MGB, 5-TAMRA (5-Carboxytetramethylrhodamine), Black Hole Quencher™, or ZEN/Iowa Black™ quenchers. Probes can also feature 6-Ccrboxyfluorescein (6-FAM), VIC®, ABY®, NED®, SUN®, JUN®, Cy3, Cy5, and Cy5.5 fluorophores. Probes can be composed of standard deoxyribonucleic bases or a mixture of locked-nucleic acid modified bases and standard deoxyribonucleic bases.

In another implementation, podocytes can be encapsulated and segmented by means of a custom-made of commercially available droplet generator (such as QX-200® from Bio-Rad) at levels in which each droplet contains a single cell (podocyte). Podocytes can then be subject to RT-PCR for podocyte-specific markers and enumerated by a droplet counter, e.g., a QX200® or QX600® droplet counter. These podocyte-specific mRNA markers can include, but are not limited to, primer/probe sets capable of detecting expression of nephrin, podocin, podocalyxin, synaptopodin, and podoplanin.

In another implementation, the podocytes are subjected to additional downstream analysis where they are interrogated for genetic mutations, gene or protein expression, as well as culturing and drug discovery.

### Predictive Evaluation of Suspected Preeclampsia

The podocyte isolation methods disclosed herein have been demonstrated to provide a significant predictive value in identifying individuals at risk of developing preeclampsia. For example, the new methods can be used during early pregnancy, e.g., after 5, 10, 15, or 20 weeks of gestational age. The new methods can serve as a compliment to existing clinical criteria for diagnosing preeclampsia, enabling healthcare providers to intervene promptly and optimize maternal and fetal outcomes. Women who are determined to be at high risk using the methods disclosed herein can be monitored more closely, or hospitalized, to provide treatment to reduce the symptoms of preeclampsia like high blood pressure, and potential seizures and fetal distress.

Moreover, by integrating the podocyte isolation method with current diagnostic methods, individuals at low risk of preeclampsia will be effectively ruled out, leading to fewer unnecessary hospitalizations, office visits, or excess monitoring, and generating substantial cost savings for the healthcare system.

### Preventive Measure for Late-Onset Preeclampsia

More than 60% of the preeclampsia cases occur at or after 37 weeks, and delivery is recommended for women with preeclampsia diagnosed at or beyond 37 weeks to achieve the best maternal and neonatal outcomes. The new podocyte isolation methods disclosed herein demonstrate high specificity and PPV, and enable prediction months earlier than possible with conventional methods of diagnosis. This predictive capability supports preventive measures where individuals at high risk of preeclampsia can be recommended for delivery before the onset of the condition, effectively averting potential cases. As a result, implementing such preventive measures may lead to a sequential reduction in the overall preeclampsia rate. This proactive approach not only improves individual health outcomes but also reduces healthcare costs associated with managing preeclampsia-related complications.

### Screening for Preeclampsia at Early Gestational Age

Screening for preeclampsia at early gestational age poses a significant challenge, with current methods relying on cumbersome risk factor criteria exhibiting a low PPV of less than 20%. The initiation of aspirin prophylaxis between 12 weeks and 28 weeks gestational age has been shown to effectively reduce the incidence of preeclampsia. However, there is currently no single reliable tool for early screening. The podocyte isolation methods described herein offer strong potential for early screening for preeclampsia. By integrating podocyte counts into routine prenatal screening protocols, healthcare providers can identify pregnant individuals at risk of developing preeclampsia and initiate timely prophylaxis to mitigate adverse outcomes, while ruling out candidates who would unnecessarily expose to the prophylaxis and not benefit.

### EXAMPLE

The disclosure is further described in the following example, which does not limit the scope of the invention described in the claims.

The aim of this example was to assess the effectiveness of the podocyte isolation methods described herein in capturing podocytes from urine samples of pregnant woman, to confirm whether the isolated podocyte count can be used as a biomarker for preeclampsia, and to evaluate the predictive capability for identifying individuals at risk of developing preeclampsia.

### Sample Collection

A total of 21 urine samples were obtained from women clinically diagnosed with either preeclampsia or gestational hypertension, constituting the case group, while an additional 21 control samples were collected from individuals without preeclampsia at the time of collection. All samples were obtained during the third trimester, prior to delivery. Control participants were matched to the preeclampsia group based on gestational age. The urine samples, ranging from 50 to 100 mL in volume, were promptly refrigerated upon collection to maintain sample integrity, and processed within 24-48 hours. Upon reception, each urine specimen underwent careful partitioning, with aliquots of 5 mL or 10 mL designated for podocyte isolation procedures. This process ensured consistency and reproducibility in subsequent analyses and allowed for the accurate assessment of podocyte numbers as a biomarker for preeclampsia.

### Preparation of Microfluidic System and Antibody-Conjugated Magnetic Bead Cocktail

The microfluidic system, characterized by a cartridge that contains a silicon chip with a super-hydrophilic surface and through holes, is illustrated in schematic form in FIG. 1, and has been previously described in, for example, U.S. Pat. Nos. 11,478,797 and 11,077,439. The methods of preparing and using these microfluidic systems are described in these patents. Specifically, the silicon microchip as used herein featured a super-hydrophilic PEG-coated surface layer and micropores (through holes), with a diameter of 6-8 μm, across the chip. Two ¼″ N52 neodymium permanent block magnets were placed underneath the microfluidic cartridge to provide the magnetic field.

To pass the sample through the microchannel in the cartridge, the inlet was connected to the sample tube, and the outlet was connected to a peristaltic pump to generate the flow using vacuum from the outlet side. In the meantime, two block magnets are placed underneath the cartridge to create the perpendicular magnetic field for capturing.

Primary antibodies targeting podocytes, specifically anti-podocin, anti-podoplanin, and anti-podocalyxin, were initially bound to PEG linkers with a molecular weight of 20 kDa. The biotinylated NHS-PEG was first dissolved in a 0.5 mM concentration solution, then conjugated with the primary antibodies at a 30-fold molar excess. The mixture was then incubated for 1 hour at room temperature, followed by filtration using a 50K MWCO protein concentrator and centrifuge at 12,000 g for 10 minutes and repeated twice.

With a typical ratio of 1 μg of primary antibody per 40 μg of beads, the antibody-PEG-biotin complexes were conjugated with streptavidin-coated superparamagnetic beads with 1 μm diameter, by incubating on a rotator for 1 hour at room temperature. The mixture was then rinsed three times with PBS on the magnetic stand, and finally resuspended in PBS, with a 1:10 dilution of the original magnetic bead concentration. This provided a “cocktail” of antibody-magnetic beads in which each bead is bound to one type of antibody, and the cocktail included a set of magnetic beads that are each bound to an anti-podocin antibody, a second set of magnetic beads that are each bound to an anti-podoplanin antibody, and a third set of magnetic beans that are each bound to anti-podocalyxin antibodies. Any such cocktail will also contain many magnetic beads that are not bound to any antibodies, but they are removed from the liquid samples by the systems described herein.

### Podocyte Capture, Detection, and Immunofluorescence Quantification

Each urine sample was first filtered using a 70 μm cell strainer to remove large crystals and particles, followed by centrifugation at 800 g for 10 minutes. Subsequently, the cell pellet was resuspended in 1 mL of 1% BSA-PBS solution. Then, a pre-prepared cocktail of antibody-beads targeting podocin, podoplanin, and podocalyxin (10 μL each) was added into the sample and incubated on a rotator for 1.5 hours at room temperature. After incubation, the sample was pumped into the microfluidic channel of the microfluidic cartridge at a controlled flow rate of 0.5 mL/min, followed by a PBS wash. Two N52 neodymium permanent block magnets were placed beneath the microfluidic cartridge for podocyte capturing and immunofluorescence quantification, thereby holding the captured podocytes in place on the microchip throughout the whole process. The captured cells were fixed on the microchip with 4% PFA solution in PBS for 5 minutes and washed.

Following fixation, the captured cells were stained in situ on the surface of the microchip with fluorescent dyes. Initially, 0.5 μg of anti-synaptopodin monoclonal antibody was incubated with 1 μg of Alexa Fluor 488-labeled secondary antibody in 0.5 mL PBS for 1 hour at room temperature, then filtered with a 0.65 μm filter, centrifuged at 2000 g for 2 minutes, and resuspended into 0.5 mL 1% BSA-PBS. Subsequently, the mixture was introduced into the microfluidic channel of the cartridge and incubated overnight in fridge.

Following thorough washing with PBS, negative immunofluorescent staining was employed to identify any white blood cells on the microchip. Specifically, 5 μL of anti-human CD45-PE in 0.5 mL 1% BSA-PBS was introduced into the microfluidic channel of the cartridge and incubated for 10 minutes at room temperature. After washing with PBS, 0.5 μL of 4,6-diamidino-2-phenylindole (DAPI) in 0.5 mL 1× perm wash buffer was added into the microfluidic channel and again incubated for 10 minutes at room temperature to label nucleated cells, followed by a thorough wash of PBS.

Finally, the microfluidic cartridge containing the microchip with the fluorescently labeled cells was scanned using a fluorescence microscope or EVOS® cell imaging systems (ThermoFisher) for cell identification. Cells stained positive for both synaptopodin and DAPI, and negative for CD45 were counted as podocytes. The number of podocytes is expressed as cells/10 mL urine.

### Statistical Analyses

Sensitivity and specificity were calculated based on the isolated podocyte numbers obtained from the case-control study. Subsequently, cutoff numbers were determined to optimize predictive accuracy under different application scenarios. Positive predictive value (PPV) and negative predictive value (NPV) curves were derived and generated against prevalence. Receiver-operating characteristic (ROC) curve analysis was performed, and the area under the curve (AUC) was computed to evaluate the predictive performance of the podocyte count as a biomarker for preeclampsia. P values were conducted using two-tailed tests, and a significance level of P<0.05 was defined as statistically significant.

### Validation Cohort Study

For the validation cohort study, 7 participants in the second trimester of pregnancy were enrolled, with no preeclampsia diagnosed at the time of sample collection. Urine samples were collected from each participant, and the same podocyte isolation and quantification method utilized in the case-control study was employed to determine the podocyte count. The cutoff number established in the controlled study was applied to assess the predictive accuracy of podocyte counts for preeclampsia, categorizing participants into two groups: low risk and high risk of developing preeclampsia.

### Results

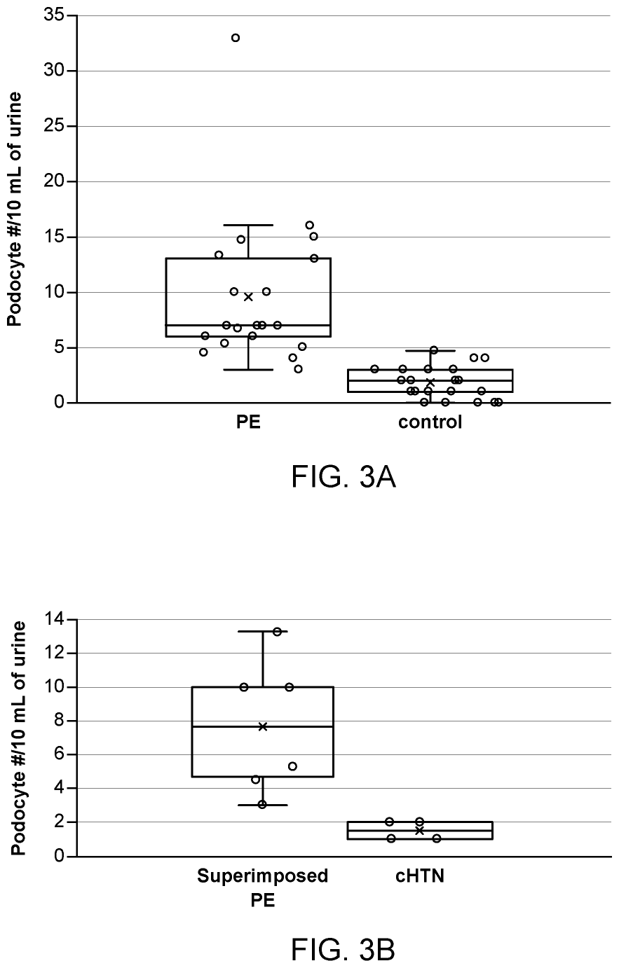

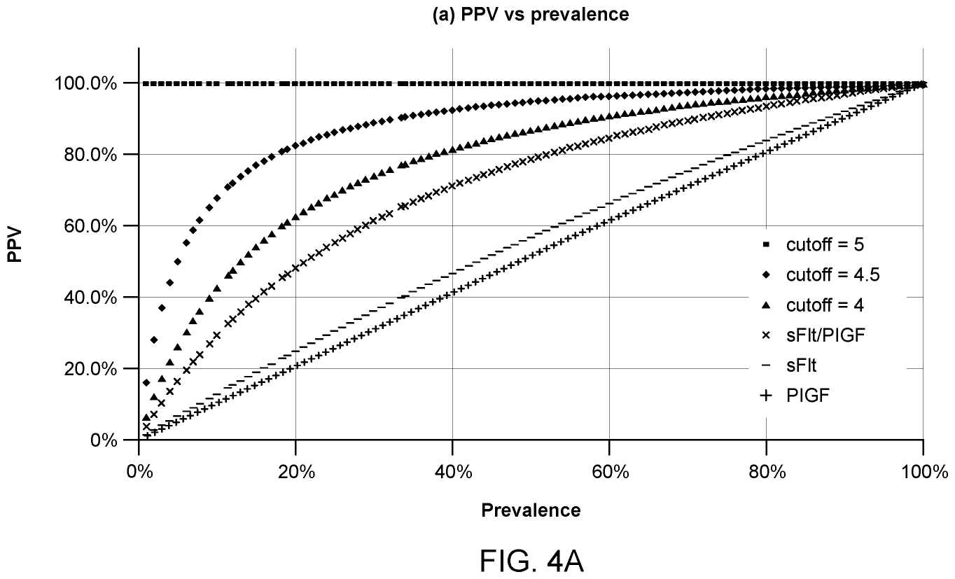

Table 1 below summarizes the gestational age, clinical characteristics, and isolated podocyte numbers for participants in the case-control study. FIG. 3A illustrates the number of podocytes isolated from each sample among the study groups. Compared to the control group (which typically had fewer than 5 podocytes, with the majority having 3 or fewer podocytes), podocyte count was significantly higher (from about 3-5 to and as high as over 30) in women diagnosed with preeclampsia and gestational hypertension (PE).

Furthermore, as shown in FIG. 3B, this number of isolated podocytes was also significantly higher in women with superimposed preeclampsia (n=6, with over 3 podocytes) than those with chronic hypertension (n=4, with two or fewer podocytes).

Based on this data, the podocyte isolation method described herein demonstrates utility in facilitating a differential diagnosis between preeclampsia and chronic hypertension, which has been difficult using conventional methods of diagnosis.

TABLE 1
Case-Control Study Results

| | Preeclampsia (PE) | | | Control | | |
|---|---|---|---|---|---|---|
| | GA | Podocyte #/ | Clinical | GA | Podocyte #/ | Clinical |
| # | (weeks) | 10 mL urine | characteristic | (weeks) | 10 mL urine | characteristic |
| 1 | 35 | 5.3 | superimposed PE | 34 | 0 | |
| 2 | 35 | 33 | | 35 | 0 | |
| 3 | 32 | 16 | | 34 | 1 | cHTN and prediabetes |
| 4 | 31 | 7 | PE with SF | 36 | 0 | |
| 5 | 36 | 4 | | 35 | 2 | cHTN |
| 6 | 37 | 4.5 | superimposed PE | 36 | 3 | |
| | | | with SF, cHTN | | | |
| 7 | 34 | 14.7 | | 34 | 2 | h/o PE in prior |
| | | | | | | pregnancy |
| 8 | 30 | 10 | superimposed PE | 35 | 4.7 | |
| | | | with SF, cHTN | | | |
| 9 | 34 | 7 | | 34 | 4 | |
| 10 | 33 | 6 | | 36 | 0 | |
| 11 | 36 | 15 | | 34 | 1 | h/o HELLP in prior |
| | | | | | | pregnancy |
| 12 | 33 | 3 | superimposed | 36 | 2 | |
| | | | PE, cHTN | | | |
| 13 | 35 | 13.3 | superimposed | 35 | 3 | |
| | | | PE, cHTN | | | |
| 14 | 40 | 7 | PE with SF | 34 | 1 | cHTN, h/o of early PE |
| | | | | | | with preterm delivery |
| 15 | 35 | 7 | | 36 | 4 | |
| 16 | 37 | 13 | | 34 | 2 | “white coat HTN”, |
| | | | | | | h/o of PE with SF in |
| | | | | | | prior pregnancy |
| 17 | 33 | 6.7 | PE with SF | 34 | 3 | |
| 18 | 34 | 6 | PE with SF | 35 | 1 | |
| 19 | 32 | 7 | gHTN | 34 | 3 | |
| 20 | 37 | 10 | superimposed | 34 | 1 | |
| | | | PE, cHTN | | | |
| 21 | 32 | 5 | PE with SF | 34 | 0 | |

| | |
|---|---|
| | Podocyte #/ |
| Gestational age | 10 mL urine |

| | mean | | mean | |
|---|---|---|---|---|
| | (±SD) | p value | (±SD) | p value |
| PE (n = 21) | 34.8 (2.3) | 0.62 | 9.5 (6.5) | <0.001 |
| Control (n = 21) | 35.1 (0.9) | | 1.8 (1.4) | |

Abbreviations: cHTN, chronic hypertension; gHTN, gestational hypertension, superimposed PE, PE patient with preexisting cHTN; SF, severe feature.

### Test Characteristics and Cutoff Numbers

Sensitivity, specificity, and F1 score analyses, detailed in Table 2, below, were performed based on the findings of the case-control study. A cutoff of 3 podocytes/10 mL urine exhibited 100% sensitivity, rendering it suitable for screening purposes. While a cutoff of 5 podocytes/10 mL urine demonstrated 100% specificity and can serve diagnostic and prognostic purposes.

TABLE 2
Predictive characteristics of different
cutoff numbers for preeclampsia

| Cutoff (podocyte #/ | | | |
|---|---|---|---|
| 10 mL urine) | Sensitivity | Specificity | F1 score |
| 3 | 100% | 67% | 0.86 |
| 3.5 | 95% | 86% | 0.91 |
| 4 | 95% | 86% | 0.91 |
| 4.5 | 90% | 95% | 0.93 |
| 5 | 86% | 100% | 0.92 |

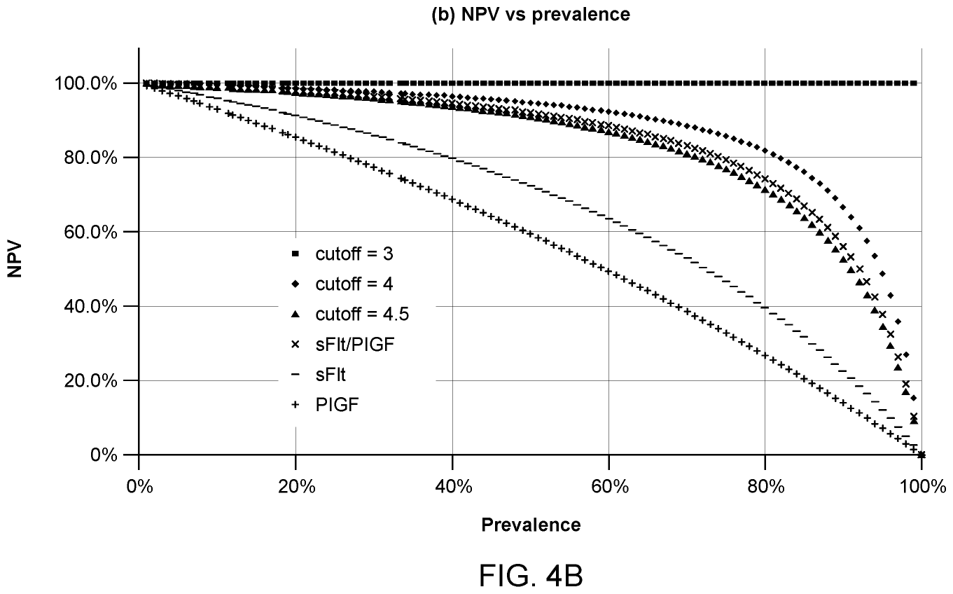

As shown in FIGS. 4A and 4B, PPV and NPV curves for various cutoff numbers were then generated against prevalence, and PPV and NPV values of maternal serum levels of sFlt, PIGF, and their ratio, were included for comparison (these values were obtained from Craici et al., “Podocyturia predates proteinuria and clinical features of preeclampsia: longitudinal prospective study.” Hypertension, 61(6), 1289-1296 (2013) and Thadhani et al., “Circulating Angiogenic factor levels in hypertensive disorders of pregnancy,” NEJM Evidence, 1(12), EVIDoa2200161 (2022)). The curves demonstrate that, by selecting appropriate cutoff numbers, the podocyte isolation method exhibits superior performance in predicting both positive and negative outcomes at different prevalence levels compared to existing biomarkers. This underscores its greater clinical utility and reliability.

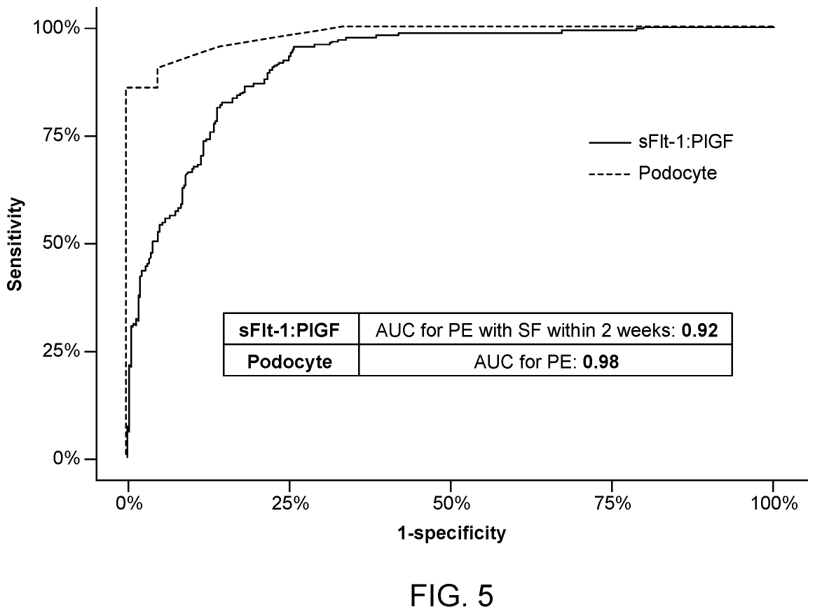

FIG. 5 illustrates the ROC curve of podocytes for preeclampsia contrasted with the ROC curve of the sFlt/PIGF result. The isolated podocyte count exhibits an AUC for preeclampsia of 0.98, surpassing that of the sFlt/PIGF (0.92), indicating superior predictive performance. The higher AUC value of podocyte means better accuracy in classifying individuals, rendering it a more reliable predictive model compared to the sFlt/PIGF. This shows the superior predictive capabilities of the new podocyte isolation method described herein.

Also note that the reported sFlt/PIGF characteristics data are intended for predicting the development of preeclampsia with severe features within two weeks from the collection of the enrollment blood sample, whereas the podocyte isolation methods described herein serve to predict the development of preeclampsia without the constraints of a specific time frame or severity level.

### Validation Cohort

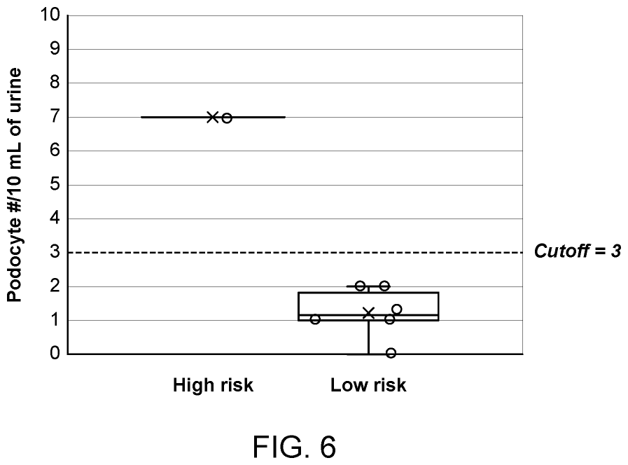

Table 3 below presents a summary of the gestational age, isolated podocyte numbers, and pregnancy outcomes for participants in the validation cohort, comprising 7 samples collected during the second trimester. The isolated podocyte numbers from each sample were graphed in FIG. 6. Given the early prediction scenario, a screening cutoff number of 3 podocytes/10 mL urine was employed, and 6 samples were categorized as low risk at 20 to 26 weeks of gestational age, while one sample exhibited high risk of preeclampsia with a count of 7 podocytes/10 mL urine at 24 weeks of gestational age.

TABLE 3
Validation cohort results

| | | | PE prediction | |
|---|---|---|---|---|
| | | | (cutoff = | |
| | GA | Podocyte #/ | 3 podocyte/ | Pregnancy outcomes |
| # | (weeks) | 10 mL urine | 10 mL urine) | confirmed upon delivery |
| 1 | 24 | 1 | Low risk | No PE |
| 2 | 24 | 1.3 | Low risk | No PE |
| 3 | 26 | 2 | Low risk | No PE |
| 4 | 20 | 0 | Low risk | No PE |
| 5 | 20 | 2 | Low risk | No PE |
| | | | | (benign intracranial |
| | | | | HTN when collected) |
| 6 | 24 | 1 | Low risk | No PE |
| 7 | 24 | 7 | High risk | PE with SF diagnosed |
| | | | | at 40 weeks GA. |

All predictions derived from the podocyte isolation were subsequently confirmed upon delivery. Of the 6 participants categorized as low risk, none developed preeclampsia, while the one with high-risk was diagnosed with preeclampsia with severe features later at 40 weeks GA, indicating 100% prediction correctness for the podocyte isolation methods described herein.

Note that the patient diagnosed with preeclampsia was not clinically identified as having preeclampsia using conventional diagnostic methods until 40 weeks of gestational age, whereas the urine sample collected for the podocyte test was obtained at 24 weeks gestational age. These results indicate a correct prediction using the podocyte isolation method 16 weeks (four months) prior to a conventional diagnosis, which shows the clear superiority of the predictive ability of the new methods disclosed herein compared to current diagnostic methods.

### OTHER EMBODIMENTS

A number of embodiments of the invention have been described. Nevertheless, it will be understood that various modifications may be made without departing from the spirit and scope of the invention. Accordingly, other embodiments are within the scope of the following claims.

1. A method of isolating podocytes from a biological sample from a pregnant woman, the method comprising
obtaining a mixture of magnetic beads and one or more different types of antibodies, wherein each of the types of antibodies bind specifically to podocytes, and wherein at least some of the magnetic beads are bound to at least one of the types of antibodies in the mixture, with each of the bound magnetic beads being bound to at least one of the antibodies;
combining the mixture of magnetic beads and antibodies with the biological sample from a pregnant woman to form a liquid sample, and incubating the liquid sample for a time and under conditions sufficient for the antibodies to bind to any podocytes that may be present in the biological sample to form podocyte-antibody-magnetic bead complexes; and
flowing the liquid sample containing podocyte-antibody-magnetic bead complexes, if any, through a microfluidic channel within a system that applies a magnetic force in a direction generally perpendicular to a direction of flow of the liquid sample with a magnetic field strength sufficient to attract the podocyte-antibody-magnetic bead complexes onto a surface of a microchip present in the fluidic chamber, and flowing remaining liquid sample out of the microfluidic channel, thereby isolating the podocytes from the biological sample.

2. The method of claim 1, wherein the antibodies are bound to a first binding partner of a binding pair, and the magnetic beads are bound to a second binding partner of the binding pair, wherein the antibodies are bound to the magnetic beads when the first binding partner binds to the second binding partner.

3. The method of claim 1, wherein the antibodies are bound to the magnetic beads or to a first binding partner via a linker molecule.

4. The method of claim 3, wherein the linker molecule comprises polyethylene glycol (PEG).

5. The method of claim 3, wherein the linker molecule has a molecular weight of about 5 kDa to 50 kDa.

6. The method of claim 1, wherein the antibodies bind specifically to podocin, podoplanin, or podocalyxin.

7. The method of claim 6, wherein the mixture of magnetic beads and antibodies comprises a first set of magnetic beads that are bound to anti-podocin antibodies, a second set of magnetic beads bound to anti-podoplanin antibodies, and a third set of magnetic beans bound to anti-podocalyxin antibodies.

8. The method of claim 1, wherein the biological sample is urine from a pregnant woman.

9. The method of claim 1, wherein the microchip comprises through holes or microwells with a through hole, wherein the through holes are smaller in diameter than podocytes, but larger than the magnetic beads, to enable excess magnetic beads to be cleared from the surface of the chip.

10. (canceled)

11. The method of claim 1, further comprising identifying which cells on the microchip are podocytes.

12-13. (canceled)

14. The method of claim 11, wherein identifying which cells on the microchip are podocytes comprises any one or more of:
positive staining of cells using a reporter group bound to an antibody that binds specifically to a podocyte-specific marker;
negative staining of cells using a reporter group bound to an antibody that specifically binds to a white blood cell marker; and
nuclear staining using a stain to label a nucleic acid.

15. The method of claim 14, comprising the use of positive staining, negative staining, and nuclear staining.

16. (canceled)

17. The method of claim 14, wherein cells that are positive for synaptopodin and DAPI and negative for CD45 are counted as podocytes.

18. The method of claim 17, wherein a presence of 3 podocytes/10 mL urine indicates a possibility that the pregnant woman may have or may develop preeclampsia during the pregnancy.

19. The method of claim 17, wherein a presence of 5 podocytes/10 mL urine indicates that the pregnant woman has or will develop preeclampsia during the pregnancy.

20-21. (canceled)

22. The method of claim 1, further comprising analyzing the isolated podocytes, by qPCR, RT-qPCR, ddPCR, next-generation sequencing (NGS), or Western blotting.

23. (canceled)

24. A method of determining a risk of a pregnant woman developing preeclampsia, the method comprising
obtaining a biological sample that may contain podocytes from a pregnant woman; and
counting podocytes in a specific volume of the biological sample,
wherein a number of podocytes above a threshold level indicates a possible risk that the pregnant woman may have or may develop preeclampsia during the pregnancy.

25. The method of claim 24, wherein the biological sample is urine, and wherein the threshold level is 3 podocytes/10 mL urine.

26. (canceled)

27. The method of claim 24, wherein the threshold level is set at 5 podocytes/10 mL urine, and wherein this threshold level indicates a high likelihood that the pregnant patient already has or will develop preeclampsia during the pregnancy.

28. The method of claim 24, wherein the biological sample is obtained at or before about 20 weeks of gestational age.

29. (canceled)
